(19) United States
(12) Patent Application Publication
(10) Pub. No.: US 2017/0326153 A1
(43) Pub. Date: Nov. 16, 2017

(54) Pharmaceutical Composition Consisting of Ceftaroline Fosamil Acetic Acid Solvate Particles Arginine Particles, Both Having A Specific Particle Size Distribution

(71) Applicant: Sandoz AG, Basel (CH)

(72) Inventor: Johannes Raneburger, Kundl (AT)

(73) Assignee: Sandoz AG, Basel (CH)

(21) Appl. No.: 15/522,687

(22) PCT Filed: Oct. 27, 2015
(86) PCT No.: PCT/EP2015/074843

§ 371 Date: Apr. 27, 2017

(30) Foreign Application Priority Data
Oct. 28, 2014 (EP) 14190751.9

(51) Int. Cl. A61K 31/546; A61K 31/675; A61K 31/67; A61K 47/18; A61K 9/14

(52) U.S. Cl. CPC A61K 31/546; A61K 47/183; A61K 9/145; A61K 31/67; A61K 31/675

(57) ABSTRACT

The invention relates to a pharmaceutical composition consisting of ceftaroline fosamil acetic acid solvate and arginine, both having a specific particle size distribution, wherein said pharmaceutical composition is stable and does not segregate into an inhomogeneous mixture.

The invention relates to a pharmaceutical composition consisting of ceftaroline fosamil acetic acid solvate particles and arginine particles, both having a specific particle size distribution, wherein said pharmaceutical composition has improved stability against segregate into an inhomogeneous mixture and has a reduced dusting tendency.

### BACKGROUND PRIOR ART

Ceftaroline fosarnil acetic acid solvate represents an advanced-generation cephalosporin antibiotic. It has activity towards methicillin-resistant Staphylococcus aureus (MRSA) and Gram-positive bacteria and is used for treating infections such as infections of skin and skin-structure as well as pneumonia.

Ceftaroline fosamil has the following structure:

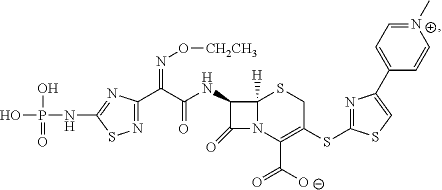 [SMILES: CCO/N=C(\C(=O)N[C@@H]1C(=O)N2C(C(=O)[O-])=C(Sc3nc(-c4cc[n+](C)cc4)cs3)CS[C@H]12)c1nsc(NP(=O)(O)O)n1]

wherein the compound is generally used in the form of its acetic acid solvate.

Ceftaroline fosamil acetic acid solvate is currently distributed in Europe under the tradename Zinforo™ which is a 600 mg powder contained in a vial, which powder is used for preparing a concentrate used for preparing infusions. In order to enhance solubility of the drug, arginine particles as solubilizer are contained in admixture with the drug.

However, such prior art mixtures of ceftaroline fosamil acetic acid solvate and arginine suffer from the problem of particle segregation/separation, meaning that the mixture is not stable and becomes inhomogeneous during processing, i.e. during preparation of the final dosage form.

The weighing of such an inhomogeneous mixture into vials is problematic and has the risk of variation in drug content in the vials. Furthermore, the prior art compositions suffer from dusting.

US 2013/0267480 A1 discloses a formulation containing the active agent “Form I NXL-104”, crystalline ceftaroline fosamil and L-Arginine (Example 9). In contrast, the present invention refers to a pharmaceutical composition consisting solely of ceftaroline fosamil acetic acid solvate particles and arginine particles to the effect that the presence of “Form I NXL-104” is excluded. The present application teaches that the particle size of both the ceftaroline fosamil acetic acid solvate particles and the arginine particles are related with a disadvantageous segregation behavior of a corresponding blend of particles. US 2013/0267480 A1 does not contain a teaching regarding how to avoid segregation of a blend of ceftaroline fosamil particles and arginine particles. Furthermore, Example 9 does neither disclose the amount of arginine particles having a size of more than 600 μm (which amount is limited by the claims of the present application), nor does it disclose the particle size distribution of the ceftaroline fosamil acetic acid solvate particles. The document does not disclose that reducing flowability reduces particle segregation in a blend of particles (see results of Example 1 of the present application).

Zhang et al. (“Effect of drug particle size on content uniformity of low-dose solid dosage forms”, International Journal of Pharmaceuticals 1997, 154, 179-183) teaches that in the context of low-dose drugs (10 μg in Table 2), larger drug particles must be reduced in size before making a homogeneous blend which provides a high content uniformity (see page 179, right column, first paragraph). As can be derived from Table 2, the smaller the particle size, the better the drug content uniformity (i.e. small Jet-milled drug is improved over the larger Bantam-milled drug). Zhang et al. did not realize that segregation of a blend of drug and excipients depends on both the particle size of the drug and the excipients. Zhang et al. therefore teaches away from the present application which particularly limits the amount of particles having a small size (see claims).

Likewise, Johnson (“Particle size of drug substance and product content uniformity—Theoretical considerations”, Formulation and Analytical Development for Low-Dose Oral Drug Products, John Wiley & Sons, Inc., US, 22 Jan. 2009, pages 49-62) teaches that large drug particles result in a poor content uniformity of low-dose drugs. Again, Johnson teaches away from the herein claimed pharmaceutical composition having a predominant amount of particles having a size of more than 100 μm and up to 500 μm, while limiting the amount of particles having a smaller size, i.e. <100 μm.

Jullien et al. (“A mechanism for particle size segregation in three dimensions”, Nature, vol. 344, 29 Mar. 1990), teaches that particle size segregation in binary mixtures increases as r (R2/R1 with R2=radius of one type of particles and R1=radius of the other type of particles) increases and x (number ratio) decreases (see page 427, left column, first paragraph). However, Jullien et al. do not deal with the more complex situation where there are not just two types of particles with ideal size, but two types of materials, each having itself a particle size distribution. Furthermore, Jullien et al. did not realize that reducing flowability of a blend (see Example 1 of the present application) reduces particle segregation. Thus, Jullien et al. do not teach to limit the amount of particles with a small particle size of <100 μm.

It is also known from “Arginine: Pharmaceutical Excipients”, Handbook of Pharmaceutical Excipients, 22 Oct. 2014, that arginine can be used as stabilizer for suppressing aggregate formation in injectable, i.e. liquid, formulations. However, the document does not address the issue related with particle segregation of a blend of particles in solid state.

EP1618894A1 discloses compositions, wherein cephem antibiotics, such as ceftaroline fosamil, are mixed with a carbonate and a basic compound, such as arginine, to produce a small amount of carbon dioxide during preparation of an infusion solution to enhance dissolution while preventing leakage of liquid during removal of said liquid from a vial by using a syringe.

In view of the above problem of segregation and dusting of mixtures of ceftaroline fosamil acetic acid solvate and arginine, there is a need for mixtures containing ceftaroline fosamil acetic acid solvate and arginine having improved stability against particle segregation and reduced dusting potential/tendency. The present invention thus aims at mixtures containing ceftaroline fosamil acetic acid solvate and arginine having improved stability and reduced dusting potential/tendency.

### SUMMARY OF THE INVENTION

It has unexpectedly been found that the stability of mixtures containing ceftaroline fosamil acetic acid solvate and arginine against particle segregation can be improved when using arginine particles having a specific particle distribution in combination with ceftaroline fosamil acetic acid solvate particles having a specific particle size distribution, wherein less than 30 wt.-% of ceftaroline fosamil acetic acid solvate particles have a particle size of up to 100 μm.

Without wishing to be bound to this theory, the inventors of the present invention believe that the small-sized particles of ceftaroline fosamil acetic acid solvate act as a lubricant/flow improver due to the specific molecular structure of ceftaroline fosamil acetic acid solvate. That is, by reducing the content of small-sized ceftaroline fosamil acetic acid solvate particles, the content of “lubricant”/“flow improver” is reduced, resulting in a non-free flowing mixture having reduced flowing characteristic and thus a reduced tendency of particle segregation during flowing/movement of the particles during the processing thereof. In other words, a homogeneous mixture prepared by mixing ceftaroline fosamil acetic acid solvate and arginine has a reduced tendency of becoming inhomogeneous during processing of said mixture. In addition, the mixtures of the invention have a reduced dusting potential/tendency.

The mixtures/pharmaceutical compositions of the invention have improved properties, which provide benefits, such as good efficacy and safety in patients, when filled into containers.

Thus, the invention relates to a pharmaceutical composition consisting of ceftaroline fosamil acetic acid solvate and arginine particles, both having a specific particle size distribution.

### DETAILED DESCRIPTION

The invention relates to a pharmaceutical composition consisting of ceftaroline fosamil acetic acid solvate and arginine particles, both having a specific particle size distribution. The pharmaceutical composition does not need to comprise additional excipients. However, it is to be understood that the benefit of reduced particle segregation might also be achieved when adding some amounts of further excipients to the mixture of ceftaroline fosamil acetic acid solvate and arginine.

Thus, the present invention refers to a pharmaceutical composition consisting of:

(i) ceftaroline fosamil acetic acid solvate particles having the following particle size distribution as determined by sieving:

0-1 wt.-% having a particle size of more than 600 μm;

0-10 wt.-% having a particle size of more than 500 μm and up to 600 μm;

60-80 wt.-% having a particle size of more than 100 μm and up to 500 μm; and

0-30 wt.-% having a particle size of up to 100 μm; preferably 0 wt.-% of ceftaroline fosamil acetic acid solvate particles have a size above 800 μm, above 750 μm, above 700 μm or above 650 μm; and

(ii) arginine particles having the following particle size distribution as determined by sieving: 0-5 wt.-% having a particle size of more than 600 μm, preferably 0-5 wt.-% having a particle size of more than 500 μm, more preferably 0-5 wt.-% having a particle size of more than 100 μm; wherein said weight percentage is based on the total amount of arginine particles, preferably 0 wt.-% of arginine particles have a size of above 800 μm, above 700 μm, above 600 μm, above 500 μm, above 400 μm, above 300 μm, above 200 μm, above 180 μm or above 150 μm, and/or wherein the arginine particles preferably have a Gaussian particle size distribution;

wherein ceftaroline fosamil acetic acid solvate is contained in the pharmaceutical composition in an amount in the range of from 54 wt.-% to 66 wt.-%, based on the total weight of the pharmaceutical composition.

In the context of the present invention, all given weight percentages with respect to the particle size distribution of ceftaroline fosamil acetic acid solvate particles are based on the total amount of ceftaroline fosamil acetic acid solvate particles, while not considering the weight of arginine.

The ceftaroline fosamil acetic acid solvate particles consist of ceftaroline fosamil acetic acid solvate only, i.e. the particles do not represent granules or pellets prepared by using excipients. The purity of ceftaroline fosamil acetic acid solvate typically is above 97 wt.-%, such as >97.5 wt.-%, >98 wt.-%, >98.5 wt.-%, >99 wt.-%, or 99.5 wt.-%. The ceftaroline fosamil acetic acid solvate preferably is a monohydrate.

The method for determining the particle size distribution of the ceftaroline fosamil acetic acid solvate particles and arginine particles by sieving is performed as follows:

Sieving is performed by vibration sieving, preferably using the device AS 200 Control G of RETSCH GmbH, Germany. The sieves to be used are analytical sieves in accordance with DIN-ISO 3310-1, having a diameter of 200 mm. The following sieves are used in this order from top to bottom: 600 μm sieve, 500 μm sieve, 100 μm sieve and sieve bottom.

10-20 g of sample composition are weighed in (accuracy of 0.05 g) and added on the top sieve (600 μm) and vibrated/sieved for 5-10 minutes at an amplitude of 1.5 and sieving interval of 5 seconds.

The amounts (g) of composition on the respective sieves and sieving bottom are then determined and the particle size distribution (in wt.-%) is then calculated, based on the total weight of the sample composition before sieving. The sum of the amounts of all sieves and the sieving pan must not be more than +/−2 wt.-% of the total weight of the sample composition before sieving. Otherwise, the measurement has to be repeated.

The volumetric weight mean particle size of the arginine particles is determined by using a volumetric dynamic laser light scattering method, preferably by using an instrument of Malvern Instruments, Ltd., Malvern, UK, preferably the Mastersizer 2000.

An unknown mixture of ceftaroline fosamil acetic acid solvate particles and arginine particles can be analyzed as follows: The sieving method as described above is performed with said mixture (instead of the pure ceftaroline fosamil acetic acid solvate or arginine particles). The respective fractions (more than 600 μm; more than 500 μm and up to 600 μm; more than 100 μm and up to 500 μm; and up to 100 μm) are weighed and the content of ceftaroline fosamil acetic acid solvate and arginine in each fraction is determined by HPLC (high performance liquid chromatograph) analysis.

The particle size distribution of the ceftaroline fosamil acetic acid solvate particles does not need to be a Gaussian function but can be asymmetric with fewer particles having a small size.

The ceftaroline fosamil acetic acid solvate particles contained in the pharmaceutical composition preferably have 0-20 wt.-%, further preferred 0-10 wt.-% of ceftaroline fosamil acetic acid solvate particles with a particle size of up to 100 μm.

Ceftaroline fosamil acetic acid solvate particles can be prepared as described in WO 2014/060202 (see specifically Examples 5 and 7 therein regarding the preparation of the particles of ceftaroline fosamil acetic acid solvate monohydrate) and EP1310502 (regarding the preparation of ceftaroline fosamil).

The ceftaroline fosamil acetic acid solvate particles used for preparing/providing the ceftaroline fosamil acetic acid solvate particles having the content of particles with a size of up to 100 μm can for example have a volumetric weight mean particle size of about 200 μm, such as from 100 to 300 μm as determined by dynamic laser light scattering. In other words, the ceftaroline fosamil acetic acid solvate particles have the above volumetric weight mean particle sizes before separating particles with a size of up to 100 μm or before blending with bigger sized particles. The ceftaroline fosamil acetic acid solvate particles and/or arginine particles having the desired particle size distribution can thus also be prepared by blending different types of ceftaroline fosamil acetic acid solvate particles and/or arginine particles having different particle size distributions. Another approach is to perform a step of milling or grinding to provide the desired ceftaroline fosamil acetic acid solvate particles and/or arginine particles. The ceftaroline fosamil acetic acid solvate particles can then be subjected to a step of removing particles having a size of up to 100 μm or blending with bigger-sized particles, if the amount of said particles is more than 30 wt.-%.

The arginine particles contained in the pharmaceutical composition preferably (in addition to the above specified particle distribution as determined by sieving) have a volumetric weight mean particle size in the range of from 15 to 40 μm, preferably 18 to 35 μm, as determined by volumetric dynamic laser light scattering method.

The mixture of ceftaroline fosamil acetic acid solvate particles and arginine particles is non-free flowing and has not been granulated, compacted or lyophilized.

The amount of ceftaroline fosamil acetic acid solvate contained in the pharmaceutical composition preferably can e.g. be 540-660 mg. The pharmaceutical composition preferably is in the form of a final form or final dosage form, which means that the composition is not mixed with further excipients and is not further treated by granulation, pelletization, compression, size classification or any method which influences the particle size distribution of the pharmaceutical composition. The pharmaceutical composition is in a dry state, i.e. is a dry powder composition. The pharmaceutical composition is suitable for preparing a concentrate which can be used for preparing infusions. The concentrate can be prepared as it is done with the commercial prior art product by adding a dissolution liquid, e.g. adding said dissolution liquid into a container comprising said pharmaceutical composition, for dissolving the mixture of ceftaroline fosamil acetic acid solvate and arginine. The concentration can then be added to an infusion solution.

The pharmaceutical composition of the invention has improved stability against particle segregation of ceftaroline fosamil acetic acid solvate particles and arginine particles and can be filled into vials without particle segregation.

The term stable means that the mixture/pharmaceutical composition is stable against particle segregation. Stability can be tested as follows: A mixture of ceftaroline fosamil acetic acid solvate particles and arginine particles is prepared by mixing (e.g. in a Diosna P1-6 mixer) for some minutes until a homogeneous mixture can be expected to be obtained. After turning of the mixing device, samples of the mixture are filled into vials and the ceftaroline fosamil acetic acid solvate content is determined and compared with the theoretical content that would be obtained with a homogeneous mixture, i.e. the theoretical content in a homogeneous mixture is 100%. Then, the relative standard deviation (%) can be determined (see example part below). The higher the degree of particle segregation, the higher the relative standard deviation. Preferably, the relative standard deviation is below 3%, such as below 2.5%, below 2.0%, below 1.5%, or below 1%.

The invention also refers to a container, preferably glass vial, containing 900-1100 mg of the pharmaceutical composition of the invention.

The container can be suitable for adding a dissolution liquid into the container and removing a (concentrate) solution comprising ceftaroline fosamil acetic acid solvate and arginine. The container can e.g. have a volume in a range of from 5 to 45 mL.

The invention also refers to a process for preparing a pharmaceutical composition of the invention, comprising or consisting of the steps of:

(i) providing ceftaroline fosamil acetic acid solvate particles;

(ii) if said ceftaroline fosamil acetic acid solvate particles of step (i) have more than 30 wt.-% of particles having a size of up to 100 μm, then performing a step of:

(a) removing at least a portion of said ceftaroline fosamil acetic acid solvate particles having a size of up to 100 μm; or

(b) blending ceftaroline fosamil acetic acid solvate particles having less than 30 wt.-% of particles having a size of up to 100 μm with the ceftaroline fosamil acetic acid solvate particles of step (i),

wherein steps (a) and (b) provide ceftaroline fosamil acetic acid solvate particles comprising 0-30 wt.-% of particles having a size of up to 100 μm; and

(iii) mixing the ceftaroline fosamil acetic acid solvate particles comprising 0-30 wt.-% of particles having a size of up to 100 μm as provided in step (i) or as obtained in step (ii) with arginine particles having the following particle size distribution as determined by sieving: 0-5 wt.-% having a particle size of more than 600 μm, wherein said weight percentage is based on the total amount of arginine particles, thereby obtaining said pharmaceutical composition.

Step (ii) can comprise sieving the composition, e.g. with a 100 μm-sieve, or performing an air separation (by using e.g. fine classifier devices available from NETSCH, Germany) in order to remove at least a portion of said ceftaroline fosamil acetic acid solvate particles having a size of up to 100 μm. The “mixing step” in step (iv) is performed until a homogeneous mixture is obtained.

The process can further comprise a step of removing ceftaroline fosamil acetic acid solvate particles having a particle size of more than 600 μm in order to reduce the particles having a particle size of more than 600 μm to an amount of 0-1 wt.-%.

The ceftaroline fosamil acetic acid solvate particles provided in step (i) can have the following particle size distribution:

0-1 wt.-% having a particle size of more than 600 μm;

0-10 wt.-% having a particle size of more than 500 μm and up to 600 μm; 60-80 wt.-% having a particle size of more than 100 μm and up to 500 μm; and

more than 30 wt.-% having a particle size of up to 100 μm.

The ceftaroline fosamil acetic acid solvate particles provided in step (i) or (ii) can have the following particle size distribution as determined by sieving:

0-1 wt.-% having a particle size of more than 600 μm;

0-10 wt.-% having a particle size of more than 500 μm and up to 600 μm;

60-80 wt.-% having a particle size of more than 100 μm and up to 500 μm; and

0-30 wt.-% having a particle size of up to 100 μm.

The invention also refers to a process for preparing a container of the invention, comprising or consisting of the steps of providing a pharmaceutical composition of the invention, preferably by performing the process of the invention, and filling said pharmaceutical composition into a container. The step of filling said pharmaceutical composition into a container comprises metering the desired amount thereof.

The pharmaceutical composition of the invention or the container of the invention comprising said pharmaceutical composition is for use in a method of treating bacterial infections, preferably, bacterial infections of skin and skin-structure as well as pneumonia.

### EXAMPLES

The following examples describe the present invention in detail, but are not to be construed to be in any way limiting for the present invention.

### Example-1

Sample size: 100 g; 66.8 g of ceftaroline fosamil acetic acid solvate (purity 90.5%) having 10 wt.-% particles <100 μm; 39.6 g arginine having a volumetric mean of 22 μm and less than 5 wt.-% of particles having a size above 100 μm.

Both components are mixed in a Diosna P1-6 mixer (which has a capacity of 500 ml) for 20 minutes, mixing condition: 150 rpm. Then, the mixture is filled into 20H vials, wherein 1.064 g are filled into each vial. The obtained mixture was determined to be non-free flowing.

### Example-2

A further example and comparative example were prepared, wherein the two different types (i.e. differing by their particle size distribution) of ceftaroline fosamil acetic acid solvate had the content of fine particles as indicated in the below table. The ceftaroline fosamil acetic acid solvate was mixed with arginine having less than 5 wt.-% of particles having a size above 100 μm and the obtained mixtures were filled into vials and the drug content was determined, wherein the comparative example (having a higher content of ceftaroline fosamil acetic acid solvate particles with a size below 100 μm) had a higher relative standard variation, i.e. showed the disadvantage of having a higher degree of variation in the drug content per vial.

| | | | RSD, |
|---|---|---|---|
| | fine particles of ceftaroline | drug content | relative standard |
| | acetic acid solvate <100 μm | n = 3 vials | deviation (%) |
| LotA | 35% | 91; 101; 108% | 5.20% |
| LotB | 7% | 101; 101; 104.9% | 1.33% |

### CITED LITERATURE

EP1310502; EP1618894; WO 2014/060202; US 2013/0267480 A1; Zhang et al. (“Effect of drug particle size on content uniformity of low-dose solid dosage forms”, International Journal of Pharmaceuticals 1997, 154, 179-183); Johnson (“Particle size of drug substance and product content uniformity—Theoretical considerations”, Formulation and Analytical Development for Low-Dose Oral Drug Products, John Wiley & Sons, Inc., US, 22 Jan. 2009, pages 49-62); Jullien et al. (“A mechanism for particle size segregation in three dimensions”, Nature, vol. 344, 29 Mar. 1990); and Arginine: Pharmaceutical Excipients“, in Handbook of Pharmaceutical Excipients, 22 Oct. 2014, 5 pages.

1. Pharmaceutical composition consisting of:
(i) ceftaroline fosamil acetic acid solvate particles having the following particle size distribution as determined by sieving:
0-1 wt.-% having a particle size of more than 600 μm;
0-10 wt.-% having a particle size of more than 500 μm and up to 600 μm;
60-80 wt.-% having a particle size of more than 100 μm and up to 500 μm; and
0-30 wt.-% having a particle size of up to 100 μm; wherein said weight percentages are based on the total amount of ceftaroline fosamil acetic acid solvate particles; and
(ii) arginine particles having the following particle size distribution as determined by sieving:
0-5 wt.-% having a particle size of more than 600 μm; wherein said weight percentage is based on the total amount of arginine particles;
wherein ceftaroline fosamil acetic acid solvate is contained in the pharmaceutical composition in an amount in the range of from 54 wt.-% to 66 wt.-%, based on the total weight of the pharmaceutical composition.

2. The pharmaceutical composition according to claim 1, having 0-20 wt. % of ceftaroline fosamil acetic acid solvate particles with a particle size of up to 100 μm.

3. The pharmaceutical composition according to claim 1, wherein the arginine particles have the following particle size distribution as determined by sieving: 0-5 wt.-% having a particle size of more than 500 μm.

4. The pharmaceutical composition according to claim 1, wherein the mixture of (i) and (ii) is non-free flowing and has not been granulated, compacted or lyophilized.

5. . The pharmaceutical composition according to claim 1, wherein said ceftaroline fosamil acetic acid solvate is a monohydrate.

6. The pharmaceutical composition according to claim 1, containing an amount of ceftaroline fosamil acetic acid solvate which is 540-660 mg.

7. The pharmaceutical composition according to claim 1 which is stable against segregation of ceftaroline fosamil acetic acid solvate particles and arginine particles.

8. Container containing 900-1100 mg of the pharmaceutical composition of claim 1.

9. The container of claim 8, which is suitable for adding a dissolution liquid into the container and removing a concentrate solution comprising ceftaroline fosamil acetic acid solvate and arginine.

10. Process for preparing a pharmaceutical composition of claim 1, comprising the steps of:
(i) providing ceftaroline fosamil acetic acid solvate particles;
(ii) if said ceftaroline fosamil acetic acid solvate particles of step (i) have more than 30 wt.-% of particles having a size of up to 100 μm, then performing a step of:
(a) removing at least a portion of said ceftaroline fosamil acetic acid solvate particles having a size of up to 100 μm; or
(b) blending ceftaroline fosamil acetic acid solvate particles having less than 30 wt.-% of particles having a size of up to 100 μm with the ceftaroline fosamil acetic acid solvate particles of step (i),
wherein steps (a) and (b) provide ceftaroline fosamil acetic acid solvate particles comprising 0-30 wt.-% of particles having a size of up to 100 μm; and
(iii) mixing the ceftaroline fosamil acetic acid solvate particles comprising 0-30 wt.-% of particles having a size of up to 100 μm as provided in step (i) or as obtained in step (ii) with arginine particles having the following particle size distribution as determined by sieving: 0-5 wt.-% having a particle size of more than 600 μm, wherein said weight percentage is based on the total amount of arginine particles; thereby obtaining said pharmaceutical composition.

11. The process according to claim 10, wherein step (ii) comprises sieving the composition or performing an air separation in order to remove at least a portion of said ceftaroline fosamil acetic acid solvate particles having a size of below 100 μm.

12. The process according to claim 10, wherein the ceftaroline fosamil acetic acid solvate particles provided in step (i) have the following particle size distribution:
0-1 wt.-% having a particle size of more than 600 μm;
0-10 wt.-% having a particle size of more than 500 μm and up to 600 μm;
60-80 wt.-% having a particle size of more than 100 μm and up to 500 μm; and
more than 30 wt.-% having a particle size of up to 100 μm.

13. The process according to claim 10, wherein the ceftaroline fosamil acetic acid solvate particles provided in step (i) or (ii) have the following particle size distribution as determined by sieving:
0-1 wt.-% having a particle size of more than 600 μm;
0-10 wt.-% having a particle size of more than 500 μm and up to 600 μm;
60-80 wt.-% having a particle size of more than 100 μm and up to 500 μm; and
0-30 wt.-% having a particle size of up to 100 μm.

14-15. (canceled)

16. The pharmaceutical composition according to claim 1, having 0-10 wt.-% of ceftaroline fosamil acetic acid solvate particles with a particle size of up to 100 μm.

17. The pharmaceutical composition according to claim 1, wherein the arginine particles have the following particle size distribution as determined by sieving: 0-5 wt.-% having a particle size of more than 100 μm.

18. The pharmaceutical composition according to claim 1, wherein the arginine particles have a volumetric weight mean particle size in the range of from 18 to 35 μm, as determined by volumetric dynamic laser light scattering method.

19. A glass vial containing 900-1100 mg of the pharmaceutical composition of claim 1.

20. A method of treating bacterial infections of skin and skin-structure as well as pneumonia comprising administering the pharmaceutical composition of claim 1.

21. The pharmaceutical composition according to claim 1, wherein the arginine particles have a volumetric weight mean particle size in the range of from 15 to 40 μm, as determined by volumetric dynamic laser light scattering method.

22. The pharmaceutical composition according to claim 1, wherein the pharmaceutical composition is in the form of a final dosage form.
